(19) United States
(12) Patent Application Publication
(10) Pub. No.: US 2003/0125881 A1
(43) Pub. Date: Jul. 3, 2003

(54) Apparatus and method for plasmapheresis

(76) Inventor: Vincent J. Ryan, Davenport, IA (US)

Correspondence Address: SPENCER, FANE, BRITT & BROWNE, 1000 WALNUT STREET, SUITE 1400, KANSAS CITY, MO, 64106-2140, US

(21) Appl. No.: 10/302,682

(22) Filed: Nov. 22, 2002

Related U.S. Application Data
(60) Provisional application No. 60/337,215, filed on Nov. 26, 2001.

(51) Int. Cl. G01N 33/48; G01F 1/00

(52) U.S. Cl. 702/19; 702/45

(57) ABSTRACT

A method and apparatus for obtaining the plasma portion from whole blood is provided which utilized a processor controlled plasmapheresis device and/or blood input pump to withdraw donor-weight specific plasma volumes and blood volumes from an individual.

### CROSS REFERENCE TO RELATED APPLICATION

Reference is made to the concurrently pending U.S. provisional application of the present inventor, Vincent Ryan, serial No. 60/337,215 and to which a claim of priority is made by this application and which specification is incorporated herein by reference.

### FIELD OF THE INVENTION

The present invention relates to the extraction of plasma from whole blood. In particular, the present invention provides an apparatus and a method of obtaining plasma from a donor based upon the specific weight of the particular donor and which weight is entered into a computer processor to determine the individual whole blood volume that can be removed from the donor and/or the volume of plasma that can be extracted from the donor during a donation session. In the apparatus, a controller in communication with the processor directs the functions of the blood extraction or plasmapheresis unit to obtain the determined volume of whole blood and/or volume of plasma from the donor.

### BACKGROUND OF THE INVENTION

Plasma is a straw-colored, clear liquid which is 90% water. Plasma contains dissolved salts and minerals such as calcium, sodium, magnesium and potassium. It is in the plasma portion of the blood that antibodies are transported to infection sites and to disease agents within the human body. A number of human proteins can be isolated from plasma including human prothrombin (factor II), human alpha-thrombin (factor IIa), gamma thrombin, human factor V, human factor VII (proconvertin), human factor VIIa, human protein C, human protein S, human factor IX, human factor X, and a multitude of other proteins including the globulins. In general, plasma can be defined as the liquid portion of blood in which the particulate components of blood are suspended.

Approximately 45% of the volume blood is in the form of cellular components which include red cells, leukocytes and platelets. The remaining 55% of blood is made up of plasma. Plasma is composed of approximately 90% water, 7% protein and 3% of various other organic and inorganic substances. Since blood plasma contains so many useful components, obtaining plasma from whole blood is an increasingly important endeavor. The modern processes for obtaining plasma from blood include “plasmapheresis” which, for the purposes of this application, is defined as the separation of a portion of the plasma fraction of the blood from the cellular components of the blood and which is effected by ultra filtration. It is important to appreciate that plasmapheresis, in this definition, is intended to obtain the plasma portion of blood.

### SUMMARY OF THE INVENTION

The present invention provides a method and apparatus of determining and obtaining a plasma volume from a donor utilizing a donor-specific body weight to determine the total plasma volume which safely can be taken from the donor and/or what whole blood volume is required to provide the calculated plasma portion and a blood cellular portion. The apparatus involved comprises a plasmapheresis apparatus having a controller and a computer processor functioning to calculate the plasma volume to be obtained from a donor and/or the related whole blood volume based upon the donor-specific body weight. The donor-specific body weight is entered into the processor and the determined volume to be obtained which is transferred to the controller which then directs the functioning of the plasmapheresis device.

The foregoing and other objects are intended to be illustrative of the invention and are not meant in a limiting sense. Many possible embodiments of the invention may be made and will be readily evident upon a study of the following specification and accompanying drawings comprising a part thereof. Various features and subcombinations of invention may be employed without reference to other features and subcombinations. Other objects and advantages of this invention will become apparent from the following description taken in connection with the accompanying drawings, wherein is set forth by way of illustration and example, an embodiment of this invention.

### DESCRIPTION OF THE DRAWINGS

Preferred embodiments of the invention, illustrative of the best modes in which the applicant has contemplated applying the principles, are set forth in the following description and are shown in the drawings and are particularly and distinctly pointed out and set forth in the appended claims.

FIG. 1 is a diagram showing the components of a prior art plasmapheresis unit; and

FIG. 2 is a diagram showing the components of a the plasmapheresis unit of the present invention and which includes a controller and processor for determination of the collection volumes based upon the donor-specific body weight.

### DESCRIPTION OF THE PREFERRED EMBODIMENT

As required, detailed embodiments of the present inventions are disclosed herein; however, it is to be understood that the disclosed embodiments are merely exemplary of the invention, which may be embodied in various forms. Therefore, specific structural and functional details disclosed herein are not to be interpreted as limiting, but merely as a basis for the claims and as a representative basis for teaching one skilled in the art to variously employ the present invention in virtually any appropriately detailed structure.

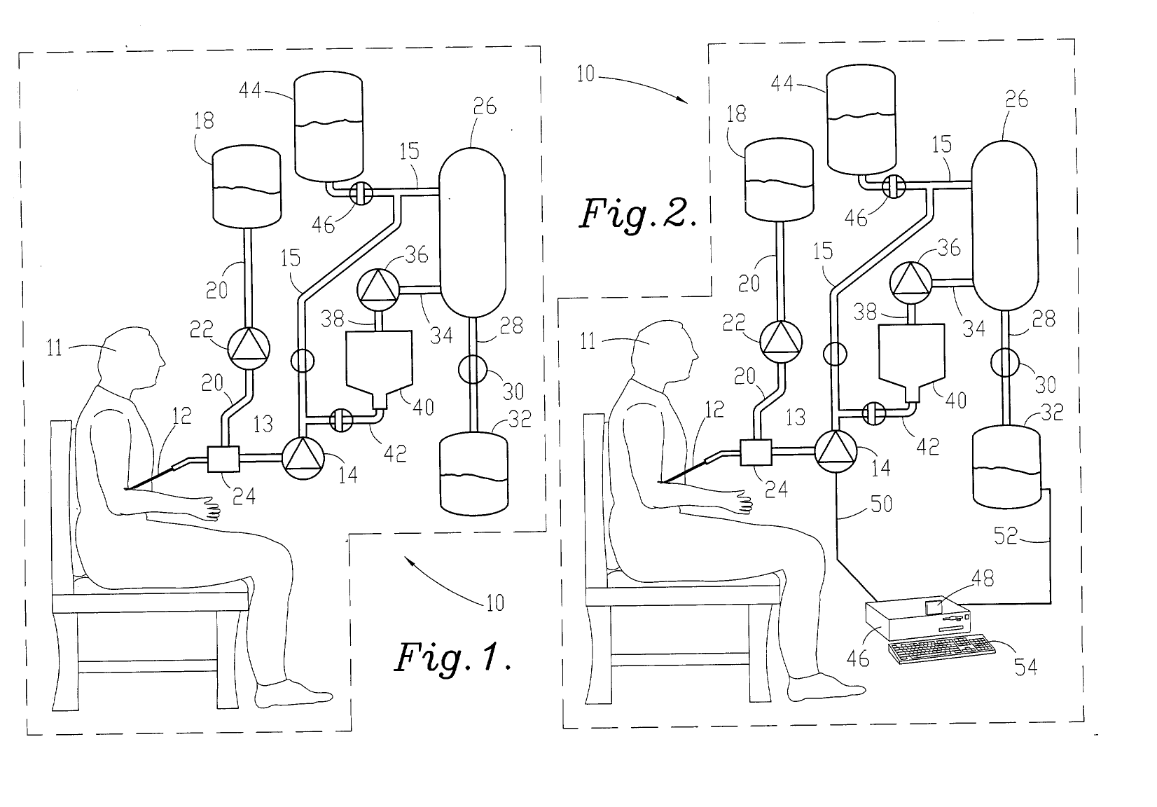

Referring now to FIG. 1, the general operation of the present day autopheresis device will be described. FIG. 1 shows in diagrammatic form a typical plasmapheresis system operating in the collection mode to pump or extract whole blood from a donor 11 via an intravenous needle 12 which has been positioned in the vein of the donor by a technician. The whole blood leaves donor 11 via needle 12 and moves through tube 13 until it contacts whole blood pump 14 which serves to continue to move the whole blood along the preseparator tube 15. Whole blood pump 14 typically will be a peristaltic pump in which tubing, such as that used for tube 13 and preseparator tube 15 is inserted. New tubing used for each donor 11 that is connected to system 10. As the whole blood is pumped from donor 11, an anticoagulant is mixed with the blood to avoid coagulation of the blood within system 10 and to reduce the chances of lysis of cellular components contained in the blood. In system 10, an anticoagulant, such as sodium citrate is contained in reservoir 18 and is pumped through anticoagulant line 20 by anticoagulant pump 22 to intravenous needle 12 where anticoagulant line 20 is joined with tube 13 by one-way valve 24. One-way valve 24 allows the mixing of the anticoagulant with the whole blood prior to movement of the whole blood through tube 13.

Still referring to FIG. 1, after the whole blood from donor 11 leaves whole blood pump 14, it passes into preseparator tube 15 and is deposited into separator 26 continuously during the course of blood extraction from donor 11. Separator 26 is a conventional plasma separation membrane device which comprises a membrane that allows the passage of the plasma component of whole blood therethrough while preventing passage of the cellular components of whole blood through the membrane. This results in separation of the plasma component of blood from the cellular components of blood. After separation, the plasma component passes through plasma tube 28 and through plasma valve 30 and is deposited in plasma reservoir 32. The cellular component of whole blood leaves separator 26 along cellular output line 34. The movement of the whole blood cellular component from separator 26 is assisted by cellular component pump 36 whereupon the whole blood cellular component is passed along line 38 until it is deposited in cellular component holding reservoir 40 until it is reinfused into donor 11 along reinfusion line 42 which passes through blood pump 14 and back into donor 11. Alternatively, if it is desired to add saline to the cellular component in reservoir 40 as it is reinfused into donor 11, saline can be added in from saline bag 44 by opening valve 46 and allowing saline to flow from reservoir 44 into the preseparator tube 15 after the collection of plasma.

In operating present day plasmapheresis devices, it is necessary for the technician operating the plasmapheresis device to determine the amount of plasma which can safely be obtained from donor 11 which in turn determines the volume of blood to be removed from a donor.

The determination of the amount of plasma which can be derived from an individual donor 11 is calculated based on the donor's body weight, hematocrit and the amount of anticoagulant used in the procedure. These factors are used in the following equation to determine the plasma collection volume which can be obtained from a particular donor. If the donor's weight is less than 175 pounds, a total blood volume of 1000 mL is obtained from the donor, and if the donor's weight is greater than 175 pounds, 1200 mL is obtained from the donor.

$$V p = \frac{( 100 - H t = AC ) \times ( V b )}{100}$$

where:

Vp=plasma collection volume in milliliters

Ht=hematocrit

AC=anticoagulant ratio

The above equation is not actually put to use on each donor, rather, a nomogram such as that shown in Table 1 is provided to the technician and on which the donor's hematocrit is located. Then, depending on the donor body weight and the percent of anticoagulant used, an expected plasma volume can be determined from the nomogram which presents the various plasma volumes that may be obtained depending upon those three factors. For example, a donor having a hematocrit of 42% and a weight of less than 175 pounds, will be considered capable of providing 660 mL of plasma volume when an anticoagulant to blood volume ratio of 8% is utilized. By comparison, if all factors remain the same, and a blood volume to anticoagulant ratio of 4% is used, a plasma volume of 620 mL can be expected from the same donor.

TABLE 1
Plasma & Anticoagulant Volume by anticoagulant ratio

| | 8% AC Ratio | | 7% AC Ratio | | 6% AC Ratio | | 5% AC Ratio | | 4% AC Ratio | |
|---|---|---|---|---|---|---|---|---|---|---|
| | Donor Weight | | Donor Weight | | Donor Weight | | Donor Weight | | Donor Weight | |
| | <175 | ≧175 | <175 | ≧175 | <175 | ≧175 | <175 | ≧175 | <175 | ≧175 |
| Hct % | lbs | lbs | lbs | lbs | lbs | lbs | lbs | lbs | lbs | lbs |
| 41 | 670 | 805 | 660 | 790 | 650 | 780 | 640 | 770 | 630 | 755 |
| 42 | 660 | 790 | 650 | 780 | 640 | 770 | 630 | 755 | 620 | 745 |
| 43 | 650 | 780 | 640 | 770 | 630 | 755 | 620 | 745 | 610 | 730 |
| 44 | 640 | 770 | 630 | 755 | 620 | 745 | 610 | 730 | 600 | 720 |
| 45 | 630 | 755 | 620 | 745 | 610 | 730 | 600 | 720 | 590 | 710 |

To simplify plasma collection procedures and to provide a guideline for plasma collections, the FDA issued a guideline on Dec. 4, 1992, titled “Volume Limits for Anticoagulant Collection of Source Plasma” which provided the simplified nomogram shown in Table 2. This nomogram can be used in plasmapheresis systems to quickly determine the plasma volume or plasma and anticoagulant (AC) volume to be expected from a donor based on the weight of the donor when a 4% solution of sodium citrate is infused into the blood at a rate to provide a 6% ratio of anticoagulant to uncoagulated 3 whole blood.

TABLE 2
FDA Guideline for Plasma Volume (mL) and Collection
Volume (Plasma Volume and Anticoagulant Volume) (mL)

| | | Plasma and AC Volume |
|---|---|---|
| Donor Weight | Plasma Volume (Weight) | (Weight) |
| 110-149 lb. | 625 mL (640 g) | 690 mL (705 g) |
| 150-174 lb. | 750 mL (770 g) | 825 mL (845 g) |
| 175 lb. and up | 800 mL (820 g) | 880 mL (900 g) |

As can be seen by examining Table 2, the FDA guideline divides donor weights into three categories of 50 pounds each. For a donor weighing between 110 and 149 pounds, a plasma volume of 625 mL from the donor can be expected based on a total collection volume of plasma and anticoagulant of 690 mL. The second category shown in Table 2, shows a donor weighing between 150 and 174 pounds and, under the conditions identified, that is, use of a 4% sodium citrate solution which is delivered at a rate to provide a 6% ratio of anticoagulant to uncoagulated blood, the plasma volume that can be collected is 750 mL based on a total volume of 825 mL of plasma and anticoagulant from a donor. As will be discussed hereinafter, this categorization of all donors into three body weights results in a substantial reduction in the amount of plasma which can be obtained from a population of individuals having a standard weight distribution. Since the same volume of blood, and therefore plasma, is derived from an individual with a weight at the bottom of each weight category as at the top of each weight category the potential for obtaining additional plasma from the larger person in each category is lost.

In Table 2 the plasma volumes obtained by use of the method of the present invention are compared to the plasma volumes which are obtained using the FDA Guidelines shown in Table 2. The volumes obtained by the method of the present invention rely on the use of the computer processor and controller of the present invention to determine the whole blood removal volume and/or plasma collection volume obtainable from each individual donor based upon each donor's particular body weight or each donor-specific body weight. By calculating individual donor volumes and controlling the blood extraction or plasmapheresis apparatus for each donor the total plasma volume that can be obtained from a population of donors can be substantially increased.

As part of the present invention, a method of specifically determining the whole blood removal volume and the associated plasma collection volume which can be obtained from that whole blood volume is determined for each donor based upon the donor-specific body weight. In addition, an apparatus is provided which allows the plasma extraction apparatus or plasmapheresis apparatus to control the particular volume of blood that is extracted from the donor and/or to determine the amount of plasma which has been collected from the donor and thereby continue or terminate the pumping of blood from the donor. To better understand the distinction provided by the present invention versus the determination of plasma volume collection as set forth in the FDA guideline of Table 2, the following comparisons are made based upon the volumes obtained by using the FDA guideline of Table 1 as compared with the volumes obtained through use of the present invention.

Referring now to Table 3, a nomogram comparison of the plasma volume which is directed to be obtained from a donor according to the FDA guidelines of Table 2 are shown in the column marked FDA Guideline. The amount of blood volume that can be obtained from a particular donor under one embodiment of the present invention is shown in the column marked Invention. In this embodiment of the present invention a factor of 5.8 milliliters per pound of donor body weight is used to determine the amount of plasma that can be extracted safely from a donor.

The factor of 5.8 milliliters per pound of donor body weight is derived from application of the data contained in the FDA issued a guideline of Dec. 4, 1992, titled “Volume Limits for Anticoagulant Collection of Source Plasma.” From the data presented in the nomogram shown of Table 2 it can be determined that the 690 mL of plasma and anticoagulant obtained from a 110 pound individual provides a ratio of 6.27 milliliters per pound of body weight. Similarly, a ratio of 5.5 is derived from the 825 milliliters of plasma and anticoagulant obtained from a 150 pound individual and a ratio of 5.03 is derived from the 880 milliliters of plasma and anticoagulant obtained from a 175 pound individual. The average of these ratios is 5.60 milliliters per pound of body weight. The factor of 5.8 milliliters per pound of body weight was selected for use with individual of the general population who are in good health and present with an acceptable hematocrit of between 38% and 54%. If it is desired to provide and additional safety factor for an individual donor a factor of less than 5.8 milliliters per pound of body weight could be used as long as their hematocrit is within the acceptable range of between 38% and 54%. In the second example nomogram of Table 4 a factor of 5.45 milliliters per pound of body weight was used as an example of a factor at the lower end of the useful range. It is this factor which is entered into processor 48 of controller 46 to determine the plasma and anticoagulant volume to be obtained from a donor of a donor-specific body weight.

As the following example demonstrates the present invention does not always provide a greater amount of plasma from a particular individual than does the nomogram method, however, over a population of donors greater amounts of plasma can be obtained. Such a case can be shown for an individual weighing 110 pounds. In an individual of 110 pounds body weight, a Total Plasma Volume (TPV) of 2,175 (mL) is present. Under the FDA guideline, a 110 pound individual would have 690 mL of plasma and anticoagulant withdrawn (the collection volume). Under the present invention, a lesser volume is obtained from the 110 pound individual, or 638 mL of plasma and anticoagulant withdrawn (the collection volume). It should be appreciated at this point that the plasma volume extracted from a 110 pound individual under the FDA guideline is the same for an individual weighing 110 pounds as for an individual weighing 145 pounds. Specifically, the same 690 mL whole blood volume is extracted from the 110 pound individual as is extracted from a 145 pound individual.

TABLE 3
Nomogram Comparison - FDA Guidelines vs. 5.8 mL/lb
All Values Except TPV are With Anticoagulant

| WEIGHT | TPV | FDA Guidelines (mL) | Invention (mL) |
|---|---|---|---|
| 110 | 2178 | 690 | 638 |
| 115 | 2277 | 690 | 667 |
| 120 | 2376 | 690 | 696 |
| 125 | 2475 | 690 | 725 |
| 130 | 2574 | 690 | 754 |
| 135 | 2673 | 690 | 783 |
| 140 | 2772 | 690 | 812 |
| 145 | 2871 | 690 | 841 |
| 150 | 2970 | 825 | 870 |
| 155 | 3069 | 825 | 899 |
| 160 | 3168 | 825 | 928 |
| 165 | 3267 | 825 | 957 |
| 170 | 3366 | 825 | 986 |
| 175 | 3465 | 880 | 1015 |
| 180 | 3564 | 880 | 1044 |
| 185 | 3663 | 880 | 1073 |
| 190 | 3762 | 880 | 1102 |
| 195 | 3861 | 880 | 1131 |
| 200 | 3960 | 880 | 1160 |
| 205 | 4059 | 880 | 1189 |
| 210 | 4158 | 880 | 1218 |
| 215 | 4257 | 880 | 1247 |
| 220 | 4356 | 880 | 1276 |
| 225 | 4455 | 880 | 1305 |
| 230 | 4554 | 880 | 1334 |
| 235 | 4653 | 880 | 1363 |
| 240 | 4752 | 880 | 1392 |
| 245 | 4851 | 880 | 1421 |
| 250 | 4950 | 880 | 1450 |

By use of the present invention method and apparatus, the amount of plasma that is removed from an individual varies by each pound of body weight attributable to the individual. For example, for the 110 pound individual, 638 mL of plasma can be removed from the individual. However, for the 145 pound individual, 841 mL of plasma can be removed from the donor. Therefore, for a population of individuals spanning the body weights of 110 pounds to 145 pounds at five pound intervals, an additional 396 mL of plasma could be safely obtained from those individuals, or an additional 56.5 mL per person. These volumes being calculated on a standard factor utilized within the processor of one embodiment of the present invention of 5.8 mL per pound of donor body weight.

A more dramatic distinction is noted when the body weights of the donors are in the higher weight class of 175 pounds and above. In the present donor population, such weights are quite common, and substantial increases in the amount of blood pumped from an individual, and therefore, the amount of plasma extracted from a population of individuals can be obtained through use of the present method and apparatus. By way of example, and still referring to Table 2, it will be appreciated that for a 175 pound individual, that under the FDA guidelines, 880 mL of plasma volume can be extracted from the individual. This same 880 mL volume is all that is taken from an individual weighing 250 pounds. In a population of sixteen individuals spanning the weight class of 175 pounds to 250 pounds at five pound increments, a total plasma volume under the FDA guidelines of 14,080 mL would be obtained from this population. By comparison, under the present invention, a total plasma volume of 19,720 mL of total blood volume could safely be extracted from this population of individuals as a result of controlling the plasmapheresis apparatus or other blood pumping apparatus to specifically account for the particular weight of each donor offering plasma. An individual weighing 175 pounds would provide only 880 mL under the FDA guidelines, whereas under the operation of the method and apparatus of the present invention, 1,015 mL could be obtained from that individual. At the high end of the weight scale, that is, an individual weighing 250 pounds, the FDA guidelines would still only remove 800 mL of blood volume from that individual, whereas the present invention would allow over 60% more blood volume to be safely removed from the individual, or 1,450 mL.

Referring now to Table 4, a similar degree of increase can be obtained from individuals even though the volume per pound of body weight is reduced to a level of 5.45 mL per pound. Such a reduced factor could result from a desire for increased safety in response to, for example, a donor who previously has exhibited dizziness when a higher volumes per pound of body weight have been used. For the population of individuals whose weight spans 110 pounds to 145 pounds, and assuming one individual at each 5 pound increment of that weight span, the increase in plasma volume obtainable from that population is 39 mL. The results for the population of individuals spanning the weights of 175 pounds to 250 pounds at 5 pound increments using the method and apparatus of the present invention provides a substantial increase in the amount of plasma volume from which the plasma can safely be extracted. For this population of 16 individuals, a total plasma volume of 18,532 mL could be safely extracted from which the

TABLE 4
Nomogram Comparison - Current vs. 5.45 mL/lb
All Values Except TPV are With Anticoagulant

| WEIGHT | TPV | FDA Guidelines (mL) | Invention (mL) |
|---|---|---|---|
| 110 | 2178 | 690 | 630 |
| 115 | 2277 | 690 | 627 |
| 120 | 2376 | 690 | 654 |
| 125 | 2475 | 690 | 681 |
| 130 | 2574 | 690 | 709 |
| 135 | 2673 | 690 | 736 |
| 140 | 2772 | 690 | 763 |
| 145 | 2871 | 690 | 790 |
| 150 | 2970 | 825 | 818 |
| 155 | 3069 | 825 | 872 |
| 160 | 3168 | 825 | 872 |
| 165 | 3267 | 825 | 899 |
| 170 | 3366 | 825 | 927 |
| 175 | 3465 | 880 | 954 |
| 180 | 3564 | 880 | 981 |
| 185 | 3663 | 880 | 1008 |
| 190 | 3762 | 880 | 1036 |
| 195 | 3861 | 880 | 1063 |
| 200 | 3960 | 880 | 1090 |
| 205 | 4059 | 880 | 1117 |
| 210 | 4158 | 880 | 1145 |
| 215 | 4257 | 880 | 1172 |
| 220 | 4356 | 880 | 1199 |
| 225 | 4455 | 880 | 1226 |
| 230 | 4554 | 880 | 1254 |
| 235 | 4653 | 880 | 1281 |
| 240 | 4752 | 880 | 1308 |
| 245 | 4851 | 880 | 1335 |
| 250 | 4950 | 880 | 1363 |

isolated as compared to 14,080 mL under the FDA guidelines. For the sixteen individuals in this weight population, this would represent an increase of 278 mL per person. Therefore, it can be appreciated that the application of the method and apparatus of the present invention to the determination of plasma volumes that can be obtained from an individual based upon the individual's particular weight and the use of the apparatus to actually obtain that volume of plasma from the individual for can result in a substantial improvement of the amount of plasma that can be obtained from a population of individuals donating blood.

Referring now to FIG. 2, the apparatus by which the present method is performed will be described. In FIG. 2, plasmapheresis system 10 incorporates the elements of the prior art plasmapheresis system as show in FIG. 1. However, control of the plasmapheresis system 10 of the present invention is a function of controller 46 which is in communicative control of whole blood pump 14 and which control of whole blood pump 14 can be a function of either the volume of blood pumped by pump 14, or the volume of plasma contained in plasma reservoir 32. Operation of pump 14 is controlled by communication link 50 which, in a typical plasmapheresis system, would communicate the rotations of peristaltic pump 14 typically used in plasmapheresis systems to controller 46 in order to provide data on the amount of blood pumped from donor 11 by pump 14. Further control of system 10 is provided by communications link 52 which provides controller 46 with data on the volume of plasma that has arrived in plasma reservoir 32 as a result of the functioning of separator 26 on whole blood received by the pumping of pump 14. It will be appreciated by those skilled in the art that the volume of plasma in reservoir 32 can be determined by weight or by volume using optical sensors which respond to the change in light transmission through plasma reservoir 32 as various portions of plasma reservoir 32 fill with plasma. For example, an empty plasma reservoir bag 32 will have one level of light transmission whereas when the same light is transmitted through plasma reservoir bag 32 that is filled with plasma, the light transmission will differ. It is this change in light transmission which is used in prior art plasma collection devices and plasmapheresis devices to indicate what volume of plasma has been collected in reservoir 32. Alternatively, as with prior art devices, a scale can be operatively connected to plasma reservoir bag 32 to obtain a gravimetric determination of the amount of plasma contained in plasma reservoir bag 32 during the collection cycle. The data from the scale or from the optical sensor is transmitted to controller 46 and to processor 48 which monitor the amount of plasma collected. Controller 46 is responsive to processor 48 which can be any type of conventional microprocessor available for use in calculation and control functions.

In operation, an individual donor 11 will be presented for donation of plasma from their whole blood. Their cellular component of the donated blood may or may not be returned to the individual upon extraction of the plasma. The donor 11 is then weighed on a conventional scale, and the donor-specific body weight of the donor is obtained. It is the donor-specific body weight which is used within the method of the present invention to control the apparatus of the present invention. It will be appreciated that the donor-specific body weight is preferably determined to, approximately, the nearest pound, however, rounding of the donor-specific body weight to the nearest two pound interval or five pound interval, for example, would not make a substantial difference in the benefits derived from the present invention, and therefore, should be considered to be equivalents thereof. The donor-specific body weight obtained by weighing donor 11 is then provided to processor 48 by entry into a typical data entry device, such as keyboard 54 on controller 46. Processor 48 will then calculate a particular plasma volume to be extracted from the donor based upon the donor-specific body weight and the selected standard of milliliters of plasma per pound of body weight to be extracted from an individual donor according to the equation:

Vpac=DW×CV

where:

Vpac=plasma and anticoagulant collection volume in milliliters

DW=specific donor body weight

CV=collection volume in milliliters per pound of body weight

Again, it will be appreciated that the amount of plasma to be extracted per pound of body weight can be varied through use of the present method and apparatus, and that it may be considered beneficial to use a lower volume per pound of body weight for generally smaller and lighter individuals than for larger and heavier individuals.

After processor 48 has calculated the volume of plasma which may be removed from donor 11 and has calculated the expected blood volume from which will be obtained from that volume of plasma, controller 46 will operate to initiate the functioning of pump 14 by communicating with pump 14 through communication line 50. The course of pumping by pump 14, the revolutions of a peristaltic pump for example, will be monitored and will be communicated back to controller 46 by communications line 50. The rate of actual pumping can be compared with the intended volume of blood that is to be extracted from donor 11. The comparison of expected blood volume to be extracted from donor 11, and the blood volume that is actually extracted from donor 11, is a matter of a continuing calculation based upon the number of revolutions of pump 14 and the amount of blood that is pumped with each revolution of pump 14. This calculation is then continually compared with the volume of blood to be removed from donor 11 as determined by processor 48 at the beginning of the operation. Upon obtaining that goal, controller 46 can shut down pump 14 or notify the operator that the desired volume of blood has been pumped from donor 11. The expected blood volume is determined by:

$$Vwb = \frac{Vpac}{\frac{( 100 - Hct + AC )}{100}}$$

where:

Vwb=expected volume of whole blood from which the resulting volume of plasma and anticoagulant is to be obtained

Vpac=plasma and anticoagulant collection volume in milliliters

Ht=hematocrit

AC=anticoagulant ratio

A second form of volume determination can be performed by the present apparatus which is based upon the actual volume of plasma that is collected in plasma reservoir 32. In this method of operation, controller 46 of the inventive apparatus monitors communication line 52. The plasma level contained in plasma reservoir bag 32 is determined by receiving the status of conventional light transmission sensors or by weight determined by a scale such as that used in prior art plasmapheresis devices to monitor the volume of plasma which has entered reservoir 32. As the volume of plasma in reservoir 32 rises, the selected sensor, either weight or volume by light transmission, will react and register the change in plasma contained in reservoir 32. This change is communicated by communications line 52 to controller 46. Controller 46, utilizing processor 48, can compare the detected volume of plasma in reservoir bag 32 with the calculated volume of plasma that is to be extracted from the donor 11 based upon the donor-specific body weight. Upon determining that the volume has been achieved, controller 46 can cease operation of pump 14 even if the calculated total blood volume has not yet been achieved. Under this method of operation, the operator can select between terminating the blood extraction procedure based either upon the total blood volume extracted by pump 14, or upon the total plasma collected in reservoir 32.

In the foregoing description, certain terms have been used for brevity, clearness and understanding; but no unnecessary limitations are to be implied therefrom beyond the requirements of the prior art, because such terms are used for descriptive purposes and are intended to be broadly construed. Moreover, the description and illustration of the inventions is by way of example, and the scope of the inventions is not limited to the exact details shown or described.

Certain changes may be made in embodying the above invention, and in the construction thereof, without departing from the spirit and scope of the invention. It is intended that all matter contained in the above description and shown in the accompanying drawings shall be interpreted as illustrative and not meant in a limiting sense.

Having now described the features, discoveries and principles of the invention, the manner in which the inventive plasmapheresis apparatus and method are constructed and used, the characteristics of the construction, and advantageous, new and useful results obtained; the new and useful structures, devices, elements, arrangements, parts and combinations, are set forth in the appended claims.

It is also to be understood that the following claims are intended to cover all of the generic and specific features of the invention herein described, and all statements of the scope of the invention which, as a matter of language, might be said to fall therebetween.

Having thus described the invention what is claimed as new and desired to be secured by Letters Patent is as follows:

1. A method of obtaining a blood plasma fraction from a donor comprising:
determining a donor body weight,
entering said donor body weight into a processor,
calculating with said processor a donor-specific body weight collection volume to be extracted from a donor, said collection volume comprising a blood plasma component and an anticoagulant component,
pumping a blood volume from the donor with blood input pump,
adding an anticoagulant to said pumped blood volume,
separating said pumped blood volume into a blood cellular component having an anticoagulant component and said collection volume, and
detecting the accumulation of said collection volume in a receptacle until said donor-specific body weight collection volume is collected.

2. The method as claimed in claim 1 further comprising the step of adjusting said blood input pump to pump an expected volume of whole blood from the donor said expected volume of whole blood providing said plasma contained in said collection volume.

3. The method as claimed in claim 2 further comprising the steps of:
communicating said donor-specific body weight to a processor,
calculating with said processor a donor-specific body weight expected blood volume,
communicating said donor-specific body weight expected blood volume from said processor to a blood input pump controller, and
limiting said input pump operation by said pump controller to pump only said donor-specific body weight expected blood volume from the donor,

4. The method as claimed in claim 1 wherein said donor body weight is determined to the nearest pound

5. The method as claimed in claim 1 wherein said donor body weight is determined to the nearest three pounds

6. The method as claimed in claim 1 wherein said donor body weight is determined to the nearest five pounds.

7. The method as claimed in claim 1 wherein calculating step comprises multiplying said donor body weight by a factor between approximately 5 milliliters per pound of body weight and 6.2 milliliters per pound of body weight to obtain the volume in milliliters of plasma that can be extracted from the donor.

8. The method as claimed in claim 1 wherein calculating step comprises multiplying said donor body weight by a factor between approximately 5.4 milliliters per pound of body weight and 6.0 milliliters per pound of body weight to obtain the volume in milliliters of plasma that can be extracted from the donor.

9. An apparatus for obtaining a blood plasma fraction from a donor comprising:
a blood input pump means for pumping blood directly from a blood vessel,
a blood separation means connected to said input pump to fractionate blood into a plasma fraction and a blood cellular component fraction, and
a processor for determining a donor-specific body weight plasma volume to be collected from the donor.

10. The apparatus as claimed in claim 9 further comprising:
a processor for determining a donor-specific body weight blood volume to be pumped from the donor, and
an input pump controller in communication with said processor said controller operating to terminate said input pump operation upon said input pump pumping said donor-specific body weight blood volume from the donor.

11. The apparatus as claimed in claim 9 further comprising input means for enter a donor-specific body weight into said processor.

12. The apparatus as claimed in claim 9 further comprising a receptacle for receiving a blood plasma fraction from said separation means.

13. A method of obtaining a blood plasma fraction from a donor comprising:
determining a donor body weight,
entering said donor body weight into a processor,
calculating with said processor a donor-specific body weight collection volume to be obtained from a donor, said collection volume comprising a blood plasma component and an anticoagulant component,
calculating with said processor a donor-specific body weight blood volume to be extracted from a donor,
adjusting a blood input pump to pump said donor-specific body weight blood volume from the donor,
pumping said donor-specific body weight blood volume from the donor,
adding an anticoagulant to said pumped blood volume,
separating said pumped donor-specific body weight blood volume into a blood cellular component fraction and a collection volume, and
detecting the accumulation of said collection volume in a receptacle until said donor-specific body weight collection volume is collected.

14. The method as claimed in claim 13 further comprising the steps of:
communicating said donor-specific body weight to a processor,
calculating with said processor a donor-specific body weight expected blood volume,
communicating said donor-specific body weight expected blood volume from said processor to a blood input pump controller, and
limiting said input pump operation by said pump controller to pump only said donor-specific body weight expected blood volume from the donor,

15. The method as claimed in claim 13 wherein said donor body weight is determined to the nearest pound

16. The method as claimed in claim 13 wherein said donor body weight is determined to the nearest three pounds

17. The method as claimed in claim 13 wherein said donor body weight is determined to the nearest five pounds.

18. The method as claimed in claim 13 wherein calculating step comprises multiplying said donor body weight by a factor between approximately 5 milliliters per pound of body weight and 6.2 milliliters per pound of body weight to obtain the volume in milliliters of plasma that can be extracted from the donor.

19. The method as claimed in claim 13 wherein calculating step comprises multiplying said donor body weight by a factor between approximately 5.4 milliliters per pound of body weight and 6.0 milliliters per pound of body weight to obtain the volume in milliliters of plasma that can be extracted from the donor.
